(19) United States
(12) Patent Application Publication
(10) Pub. No.: US 2024/0336414 A1
(43) Pub. Date: Oct. 10, 2024

(54) EDIBLE RECEPTACLE FOR PHARMACEUTICALS

(71) Applicant: Express Scripts Strategic Development, Inc., St. Louis, MO (US)

(72) Inventor: Robert E. Hoffman, Linden, IN (US)

(21) Appl. No.: 18/131,394

(22) Filed: Apr. 6, 2023

(51) Int. Cl. B65D 65/46; A23L 29/00; A23L 29/262; A23L 29/281; A23L 29/30; A23P 20/20; A61K 9/00; B33Y 10/00; B33Y 80/00; B65B 1/02; B65B 1/04; B65B 61/02

(52) U.S. Cl. CPC B65D 65/463; A23L 29/035; A23L 29/262; A23L 29/284; A23L 29/30; A23P 20/20; A61K 9/0056; B33Y 10/00; B33Y 80/00; B65B 1/02; B65B 1/04; B65B 61/025; A23P 2020/253; B65D 2203/02

(57) ABSTRACT

An edible receptacle for pharmaceuticals, components thereof, and associated methods. The edible pharmaceutical receptacle includes a plurality of pharmaceuticals including different first and second pharmaceuticals and an edible pharmaceutical card. The edible pharmaceutical card includes a plurality of edible dosing sections joined together. The plurality of edible dosing sections include a first edible dosing section directly joined to a second edible dosing section. The first and second edible dosing sections are arranged to be manually broken apart to separate the first and second edible dosing sections from one another. The first edible dosing section contains the first pharmaceutical of the plurality of pharmaceuticals and the second edible dosing section contains the second pharmaceutical of the plurality of pharmaceuticals.

### FIELD

The present disclosure generally relates to receptacles for pharmaceuticals, and more particularly to edible receptacles for holding and containing pharmaceuticals.

### BACKGROUND

Many patients take pharmaceutical treatments that involve several different pharmaceuticals (e.g., prescription drugs) taken at regular intervals. The intervals of each pharmaceutical may not line up and can involve pharmaceuticals taken at intervals during a day and/or during a week. This can result in a complex pharmaceutical treatment regime, where different pharmaceuticals are taken at different times of the day and/or different days of the week. For example, in one day, a patient may take two different types of pharmaceuticals in the morning, another different type of pharmaceutical at lunch, and two additional pharmaceuticals (one of which can be the same as one taken in the morning) in the evening. The next day, the patient may either repeat the same regime, repeat some of the same regime or have an entirely different regime. Accordingly, the treatment regimen for a patient can become very complex depending on the number, types, and timing of the pharmaceuticals in the regimen and each patient may have a unique treatment regimen. Moreover, the efficacy of the pharmaceutical treatment may require relatively consistent compliance to the treatment regimen. That is, the efficacy of the treatment may require that the patient generally follow the treatment regimen, in terms of consistently undertaking the prescribed pharmaceutical in manner and at the times specified by the treatment regimen.

### SUMMARY

In one aspect, an edible pharmaceutical receptacle comprises a plurality of pharmaceuticals including a first pharmaceutical and second pharmaceutical. The second pharmaceutical being different than the first pharmaceutical. An edible pharmaceutical card includes a plurality of edible dosing sections joined together. The plurality of edible dosing sections includes a first edible dosing section directly joined to a second edible dosing section. The first and second edible dosing sections are arranged to be manually broken apart to separate the first and second edible dosing sections from one another. The first edible dosing section contains the first pharmaceutical of the plurality of pharmaceuticals and the second edible dosing section contains the second pharmaceutical of the plurality of pharmaceuticals.

In another aspect, a method of making an edible pharmaceutical receptacle comprises forming, via additive manufacturing, an edible base of an edible pharmaceutical receptacle using a first edible material. The method may also comprise placing a plurality of pharmaceuticals on the edible base in a plurality of pharmaceutical doses. The plurality of pharmaceutical doses includes a first pharmaceutical dose and a second pharmaceutical dose. The first pharmaceutical dose contains one or more pharmaceuticals of the plurality of pharmaceuticals. The second pharmaceutical dose contains one or more pharmaceuticals of the plurality of pharmaceuticals. The method may also comprise adding a second edible material, via additive manufacturing, to the edible base to secure the plurality of pharmaceuticals to the edible pharmaceutical receptacle in the plurality of pharmaceutical doses.

In another aspect, an edible pharmaceutical receptacle comprises a plurality of pharmaceuticals including a first pharmaceutical and second pharmaceutical. An edible pharmaceutical carrier has a bite-sized size and shape such that the edible pharmaceutical carrier can be eaten by a patient in one mouthful. The edible pharmaceutical carrier includes an enclosed first compartment, an enclosed second compartment, and a partition separating the first and second compartments. The first pharmaceutical is disposed in the first compartment and the second pharmaceutical is disposed in the second compartment.

In another aspect, a method of making an edible pharmaceutical receptacle comprises forming, via additive manufacturing, a first portion of the edible pharmaceutical receptacle using a first edible material. The first portion defines a first compartment. The method may also comprise placing a first pharmaceutical in the first compartment. The method may also comprise forming, via additive manufacturing, a second portion of the edible pharmaceutical receptacle using a second edible material. The second portion closes the first compartment.

Other objects and features will be in part apparent and in part pointed out hereinafter.

### BRIEF DESCRIPTION OF THE DRAWINGS

FIG. 1 is a fragmentary top view of an edible pharmaceutical receptacle according to one embodiment of the present disclosure;

FIG. 2 is a fragmentary top view of an edible pharmaceutical receptacle according to another embodiment of the present disclosure;

FIGS. 3A-C are perspectives showing the steps for making the edible pharmaceutical receptacle of FIG. 1;

FIGS. 4A-C are perspectives showing the steps for making the edible pharmaceutical receptacle of FIG. 2;

FIG. 5 is a perspective of an edible pharmaceutical receptacle according to another embodiment of the present disclosure;

FIG. 6 is a perspective of an edible pharmaceutical receptacle according to another embodiment of the present disclosure;

FIGS. 7A-C are perspectives showing the steps for making the edible pharmaceutical receptacle of FIG. 5; and

FIGS. 8A-E are perspectives showing the steps for making the edible pharmaceutical receptacle of FIG. 6.

Corresponding reference characters indicate corresponding parts throughout the drawings.

### DETAILED DESCRIPTION

Example edible pharmaceutical receptacles and methods of constructing the edible pharmaceutical receptacles are shown and described. The treatment of some diseases or ailments may utilize relatively complex treatment regimens that may involve multiple treatments. The treatments may include medications of various types and administration timing. For example, some treatment regimens utilize multiple different pharmaceuticals taken orally at different time intervals and over different time periods. As mentioned above, the efficacy of the pharmaceutical treatment may require relatively consistent compliance to the treatment regimen. The edible pharmaceutical receptacles of the present disclosure may generally be used to assist a person or patient who is undergoing a treatment regimen to manage the various medications (e.g., pharmaceuticals) of their pharmaceutical treatment.

The edible pharmaceutical receptacles of the present disclosure are used to hold and organize pharmaceuticals P, such as prescription drugs, supplements, vitamins, placebos (if needed), and the like, of a treatment regimen. The pharmaceuticals P may be in the form of a pill, capsule, tablet, liquid, powder, etc. The edible pharmaceutical receptacles assist those patients with complex drug treatment regimens to have a clear, systematic organization for the taking of their drugs. The edible pharmaceutical receptacles may increase compliance with the treatment regimen. In some treatment regimens there may be changes in the type of pharmaceutical P (e.g., drug) or a dosage amount at a certain time of day or a change over a longer time period, e.g., changing the drug dosage amount or drug type over the course of weeks or the regimen time period. The present disclosure describes edible pharmaceutical sections or segments that organize pharmaceuticals P over the course of the treatment. The edible pharmaceutical sections may be prepared for different dosage times (e.g., Tuesday morning, Friday evening, etc.). Each section can include the drugs for a time of day ingestion of the at least one drug. Some sections may not have a drug therein according to a treatment regime. Two or more edible pharmaceutical sections may form an edible pharmaceutical receptacle. The edible pharmaceutical receptacles can organize the treatment regimen over a long time period, e.g., one or more weeks. As used herein, the reference identifier “P” is used to designate all pharmaceuticals generally, while the reference identifier “P” followed by a subscript (e.g., “P_{A},” “P_{B},” “P_{C},” “P_{D},” etc.) is used to designate different types of pharmaceuticals.

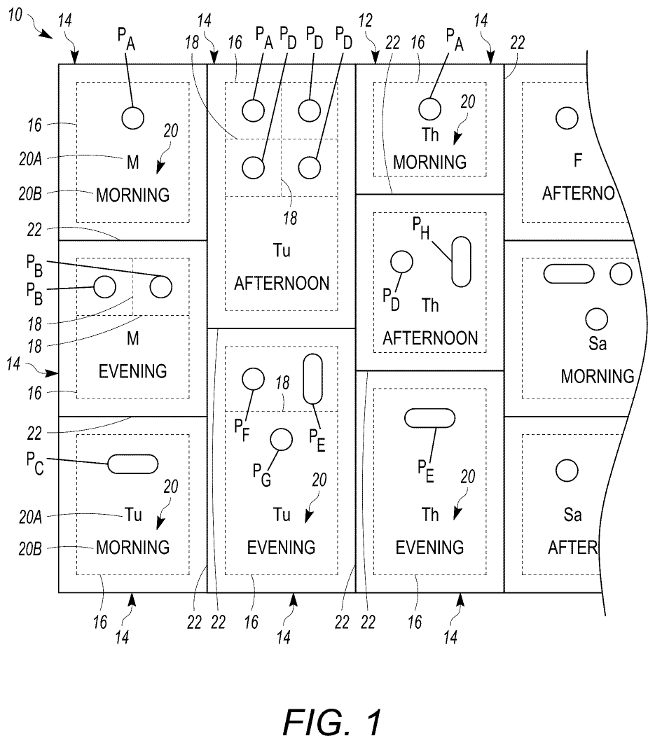

Referring to FIG. 1, one embodiment of an edible pharmaceutical receptacle (“receptacle”) of the present disclosure is generally indicated by reference numeral 10. The receptacle 10 is preferably organized by the treatment regimen (e.g., as prescribed by the medical professional, as customized for an individual patient, or otherwise). More particularly, the receptacle 10 organizes the plurality of pharmaceuticals P (e.g., a first pharmaceutical, a second pharmaceutical, a third pharmaceutical, etc.) of the treatment regimen. The pharmaceutical P can be a solid, e.g., a pill, tablet, gel cap, capsule, spanules, softgels, granules, or the like. In this embodiment, the receptacle 10 has a layout similar to conventional blister packs. The receptacle 10 includes an edible pharmaceutical card or bar 12 (broadly, an edible matrix) including a plurality of edible dosing sections or segments 14 joined together. The pharmaceutical card 12 can be a bar of an edible matrix. In an example embodiment, pharmaceutical card 12 is a cuboid. In an example embodiment, pharmaceutical card 12 is a parallelepiped. Each dosing section 14 corresponds to a dosing time (such as Monday morning, Monday noon, Monday night or the like) for the patient to take one more pharmaceuticals P of the treatment regimen. As illustrated, each dosing section corresponds to a different dosing time (e.g., time of day and day of the week) the patient is to be administered pharmaceuticals P according to the treatment regimen. The pharmaceutical card 12 can act as a multidose organizer. Preferably, each dosing section 14 contains one or more pharmaceuticals P of the treatment regimen. However, in certain embodiments, one or more dosing sections 14 may be empty (e.g., not contain any pharmaceuticals). This can be desirable in order to help build the patient's habit of taking or checking (and even eating a portion) of the receptacle at regular intervals every day (e.g., check the receptacle in the morning, afternoon, and evening every day) to help build a habit of returning to the receptacle to keep the patient adhering to the treatment regimen. In an example, embodiment, each section 14 is the same size such that the patient breaks off and ingests the same size every day or at each dosing time. This may assist with adherence so that the patient ingests the same size section 14 at each dosing time.

In the illustrated embodiment, the pharmaceuticals P in some of the dosing sections 14 are identical and in some of the dosing sections 14 are different. It is understood the types of pharmaceuticals P in the dosing sections 14 can be the same or different, and the placement of different types of pharmaceuticals (e.g., “P_{A},” “P_{B},” “P_{C},” “P_{D},” etc.) in the dosing section is based on the prescribed treatment regimen. It is also understood the dosing sections 14 can have the same or different quantities of pharmaceuticals P, again the specific quantities being based on the prescribed treatment regimen. Accordingly, the organization and placement of the different pharmaceuticals P in the individual dosing sections 14 is based on the treatment regimen of the patient. The pharmaceuticals P contained in one dosing section 14 may be a different amount relative to the pharmaceuticals P in another dosing section. In an example, the pharmaceuticals P contained in one dosing section may include one or more additional pharmaceuticals not included in another dosing section and/or the pharmaceuticals P contained in one dosing section may be the same as another dosing section. For example, in the illustrated embodiment, dosing section 14 at the top of the first column from the left contains one pharmaceutical of type P_{A}, while the dosing section 14 second from the top of the first column from the left contains two pharmaceuticals of type P_{B}. The one or more pharmaceuticals P of each dosing section 14 form a pharmaceutical dose of the treatment regimen. The pharmaceutical dose comprises the one or more pharmaceuticals P (broadly, group or set of pharmaceuticals) of the treatment regimen that are taken together at the same time. Typically, a pharmaceutical dose will include between 2-6 different pharmaceuticals. Each dosing section 14 includes one pharmaceutical dose of the treatment regimen. In an example, at least one dosing section 14 does not include a pharmaceutical dose. Accordingly, the receptacle 10 includes a plurality of pharmaceutical doses (e.g., a first pharmaceutical dose, a second pharmaceutical dose, etc.), with the number of pharmaceutical doses generally corresponding to the number of dosing sections 14.

In the embodiment illustrated in FIG. 1, each dosing section 14 includes a dosing compartment 16 sized and shaped to contain the one or more pharmaceuticals P therein (broadly, contain the pharmaceutical dose). The pharmaceuticals P are disposed in the dosing compartments 16. In one embodiment, the size and shape of each dosing compartment 16 of each dosing section 14 can be conformed to the size and/or shape of the one or more pharmaceuticals P contained in the dosing compartment. This way, the size of the dosing compartment 16, and by extension the dosing section 14, can be constructed to be just large enough to contain the pharmaceuticals P. This minimizes the amount of material needed to form the receptacle, keeping material costs down and minimizing manufacturing time. The dosing sections 14 can all be the same size and shape, different sizes and shapes (as illustrated), or a combination thereof. In one embodiment, the dosing section 14 may include one or more partitions 18 in the dosing compartment 16 to divide the dosing compartment into two or more sub-compartments. Each sub-compartment may contain none, one, or more than one pharmaceutical P. Certain pharmaceuticals P cannot be stored in contact with one another because they might react with one another, degrade, or otherwise become ineffective. The partitions 18 are used to prevent certain pharmaceuticals P from coming into contact with one another. Preferably, the dosing compartments 16 are fully enclosed and are inaccessible to prevent the pharmaceutics from being removed from the dosing section 14. For example, preferably the dosing sections 14 fully encase the pharmaceuticals P in the dosing compartments 16 and the only way to access the pharmaceuticals is by breaking the dosing section, such as what would occur when the dosing section is eaten and chewed by the patient.

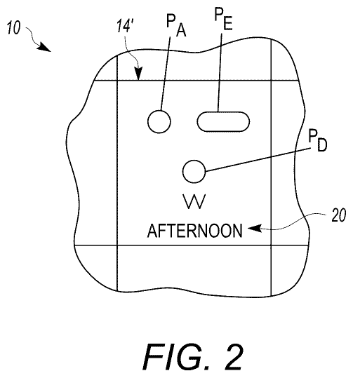

In one embodiment, the pharmaceuticals P may be embedded into the dosing section. An example of such an embodiment is generally indicated at reference numeral 14′ in FIG. 2. In this embodiment, the dosing section 14′ does not includes a dosing compartment. Instead, the pharmaceuticals P are embedded into the body or edible material of the dosing section 14′. Because the one or more pharmaceuticals P are embedded into the dosing section 14′ the pharmaceuticals are held firmly in place and kept from contacting one another. In one embodiment, the dosing section 14′ includes bumps or raised projections that correspond to the pharmaceuticals P and visually indicate the location of the pharmaceuticals in the dosing section. For example, the pharmaceuticals P may be embedded or covered by a thin layer of material that generally conforms to the size and shape of the pharmaceuticals P, thereby forming the raised bumps. Such visual indications tell the patient how may pharmaceuticals are in each dosing section 14′ and where they are located, to facilitate the patient taking every pharmaceutical in the dosing section. It is appreciated that the dosing section 14′ of FIG. 2 is generally analogous to the dosing section 14 of FIG. 1, and thus the descriptions regarding the dosing section 14 of FIG. 1 also apply to the dosing section 14′ of FIG. 2, except as clearly indicated or stated otherwise. For example, the receptacle 10 may include a plurality of dosing sections 14′ of FIG. 2. It is understood the receptacle 10 may include dosing sections 14 of FIG. 1, dosing section 14′ of FIG. 2 or a combination thereof. While the pharmaceuticals P are shown schematically in FIGS. 1 and 2, it is understood the pharmaceuticals are not visible unless the edible material of the dosing section 14, or a portion thereof, is transparent or semi-transparent thereby permitting a patient to see into the compartment 16.

Returning to FIG. 1, the receptacle 10 includes dosing indicia 20 associated with each dosing section 14. The dosing indicia 20 can be part of the dosing sections 14 or otherwise associated with the dosing sections. The dosing indicia 20 can differentiate the different dosing sections 14. In the illustrated embodiment, the dosing indicia 20 is used to indicate a specific time to take (e.g., eat) the respective dosing section and thereby take (e.g., ingest) the pharmaceuticals P contained therein. The dosing indicia 20 includes a day indicia 20A and a day time indicia 20B (broadly, at least one of a day indicia or a day time indicia). The day indicia 20A indicates a day the corresponding dosing section 14 (e.g., corresponding pharmaceutical dose) is to be taken. In the illustrated embodiment, the day indicia 20A is an abbreviation for a day of the week (e.g., M for Monday, Tu for Tuesday, W for Wednesday, Th for Thursday, F. for Friday, Sa for Saturday, and Su for Sunday). In other embodiments, the day indicia may indicate the day (e.g., day 1, day 2, day 3, etc.) of the treatment regimen. The day time indicia 20B indicates the time of day the corresponding dosing section 14 (e.g., corresponding pharmaceutical dose) is to be taken. In the illustrated embodiment, the day time indicia 20B is a word (e.g., morning, afternoon, evening, etc.) stating the time of day the dosing section 14 is to be taken. The dosing indicia 20 may be color to have a different appearance then the rest of the dosing section 14. The dosing indicia 20 may be an integral part of the dosing section or be applied (e.g., printed) thereon. Other configurations of the dosing indicia are within the scope of the present disclosure and the dosing indicia may be used to convey other types of information, such as the pharmaceutical types, pharmaceutical size (e.g., 100 mg, 200 mg, etc.), etc. Preferably, the dosing indicia 20 is constructed of an edible material, such as the same edible material of the card 12 (described below). In some embodiments, the edible material of the dosing indicia is an edible ink.

The dosing sections 14 may be arranged in generally any configuration in the card 12. In the illustrated embodiment, the dosing sections 14 are arranged in a grid-like pattern. The dosing sections 14 may be arranged sequentially in the grid-like pattern, in the order the dosing sections are to be taken. In one embodiment, the dosing sections 14 for one day may all be arranged in the same row or column of the grid-like pattern. The dosing sections 14 are joined together to form a single sheet. In one example, the single card may correspond to one week of the treatment regimen. Adjacent dosing sections 14 are joined directly together. The dosing sections 14 are arranged to be manually broken apart or separated from the rest of sheet to separate the dosing sections from one another. In the illustrated embodiment, the card 12 includes areas or lines of weakness 22, such as perforated lines, scored lines, or kerfed lines, between adjacent dosing sections 14. In an example, the lines of weakness 22 are notches in the material of the card 12 that have a reduced thickness relative to the areas of the card in which the pharmaceuticals P are stored. The lines of weakness 22 from weakened areas in the card 12 which allow a user to break apart the dosing sections 14 along the lines of weakness. The lines of weakness 22 guide the mechanical separation of the dosing sections 14. Generally, the dosing sections 14 are defined by the lines of weakness 22.

The card 12 (e.g., dosing sections 14) is constructed of one or more edible materials. In one embodiment, the edible materials are one or more of gelatin, hypromellose, makiid, sugar, edible paper/cellulose, beeswax, MonoSol's film, and combinations thereof. Other suitable edible materials are within the scope of the present disclosure. As a result of making the card 12 out of edible materials, the entire card can be orally administered to or taken by the patient. This allows the patient to break off each dosing section 14 from the rest of the card (similar to breaking a piece off a chocolate bar) and to eat the entire dosing section to take the pharmaceuticals P (e.g., broadly, the pharmaceutical dose) contained by the respective dosing section. Keeping all the pharmaceuticals P together for the pharmaceutical dose ensures greater compliance to the treatment regimen by the patient. Moreover, it is recommended or required for certain pharmaceuticals P to be taken with food and eating the edible dosing section 14 may satisfy this recommendation/requirement. The pharmaceuticals P are preferably in a form easy to ingest, such as a liquid, powder, or are chewable. However, the pharmaceuticals P may have other forms, such as swallowable tablets, as well.

When it is time for a patient to take a pharmaceutical dose of the treatment regimen, the patient simply removes the appropriate dosing section 14 from the rest of the card 12. The patient then places the dosing section 14 in their mouth and eats the dosing section 14 and the pharmaceuticals P contained therein. The patient takes all of the contents in the dosing section 14. Preferably, the dosing sections 14 have a bite-sized size and shape so that the entire dosing section can be eaten by the patient (e.g., interested into the patient's mouth) in one mouthful. If a patient were to eat the dosing section 14 in multiple bites, the breaking of the dosing section 14 into smaller bites may inadvertently release the pharmaceuticals P from the dosing section before the dosing section is completely eaten. In other words, the pharmaceuticals P may inadvertently fall out of the dosing section 14. Keeping the dosing section 14 small and bite-sized prevents such a situation from occurring.

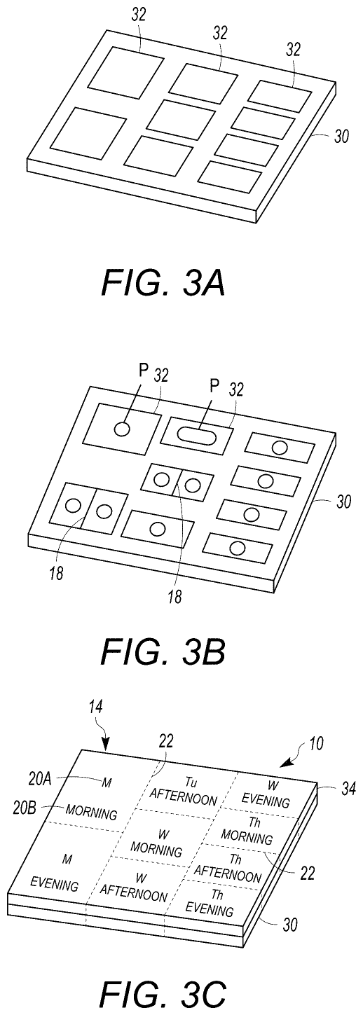

Referring to FIGS. 3A-C, one method of making the receptacle 10 of the present disclosure is generally illustrated. In this embodiment, the receptacle 10 is made using additive manufacturing (e.g., 3-dimensional (“3D”) printers), such as by building or laying down successive layers of material (e.g., edible material) on top of one another to create the object (e.g., receptacle), although other ways of making the receptacle are within the scope of the present disclosure. In the illustrated embodiment, at the start, an edible base 30 of the receptacle 10 is formed by the additive manufacturing process. For example, a food grade or pharmaceutical grade 3D printer may form the edible base 30 out of an edible material (e.g., a first edible material), such as one of the edible materials described herein. When forming the base 30, one or more depressions or recesses 32 may be formed for receiving the pharmaceuticals P. The depressions 32 will form at least a portion of the dosing compartments 16 (broadly, the depressions and dosing compartments are generally synonymous with one another). The depressions 32 may be sized and shaped to correspond to the size and shape needed to contain the specific quantity of pharmaceuticals P for each dosing section 14. In fact, because the receptacle 10 is being formed via additive manufacturing, the entire receptacle (including the size and shape of the dosing sections 14) can be customized to the treatment regimen of a particular patient. This minimizes the amount of material needed to form the receptacle, keeping material costs down and minimizing manufacturing time. The number, size, and positioning of the depressions 32 corresponds to the number, size and positioning of the dosing sections 14. If needed, any partitions 18 can be added to the base 30 by the additive manufacturing process at this time.

After the base 30 is formed, the pharmaceuticals P are placed on the base according to the treatment regimen, as illustrated in FIG. 3B. The pharmaceuticals P are arranged in the base 30 according to the pharmaceutical doses required by the treatment regimen. Placing of the pharmaceuticals P can occur manually or with one or more pharmaceutical placers/dispensers (not shown). One example of a pharmaceutical placer/dispenser includes a robotic arm with end-of-arm tooling arranged to individually carry and place the pharmaceuticals, although other configurations are within the scope of the present disclosure. After the pharmaceuticals P are placed, the rest of the receptacle 10 is formed on the base 30. Generally speaking, an edible material (e.g., a second edible material) is added to the base to secure the pharmaceuticals P to the base (broadly, receptacle 10). This is done by closing the plurality of depressions 32 with the edible material. In the illustrated embodiment, an edible cover or lid 34 is added to the base 30. In one embodiment, the edible cover 34 is formed separately of the base 30 and then attached to the base 30. One way of attaching the cover 34 the base 30 is by ultrasonic welding. In this embodiment, the cover 34 and base 30 may have corresponding projections and recesses sized and shaped to mate with one another to assist in the alignment of the cover on the base. In another embodiment, the edible cover 34 is formed directly on the base 30 using the additive manufacturing process. In either case, a food grade 3D printer may form the edible cover 34 out of an edible material (e.g., the second edible material), such as one of the edible materials described herein. The edible material of the cover 34 may be the same or different than the edible material of the base 30 (broadly, the first and second edible materials may be the same or different). In one embodiment, after the cover 34 is attached, the lines of weakness 22 are formed. This can be done by scoring or kerfing the base 30 and/or cover 34 at the appropriate locations. In one embodiment, the cover 34 and/or base 30 are formed by the additive manufacturing process with the lines of weakness 22.

The card 12 is labeled with the dosing indicia 20. The dosing indicia 20 is applied to the card 12 to correspond to the dosing sections 14 (e.g., pharmaceutical doses). In one embodiment, the dosing indicia 20 is applied (e.g., printed by a food grade 3D printer) to cover 34. This may be done before or after the cover 34 is applied to the base 30. In one embodiment, the cover 34 is formed with the dosing indicia 20 thereon (e.g., the dosing indicia is integral to the cover). For example, the food grade 3D printer may form the dosing indicia 20 as the printer is forming the cover, such as by printing additional edible material into the cover. In another embodiment, the dosing indicia 20 is applied by a grinder (controlled by a CNC machine) which removes material from the cover 34 to form the dosing indicia. Other ways of labeling the card with the dosing indicia are within the scope of the present disclosure. After the receptacle 10 is completed (FIG. 3C), the receptacle can be packaged and transferred (e.g., shipped, transported, etc.) to the patient.

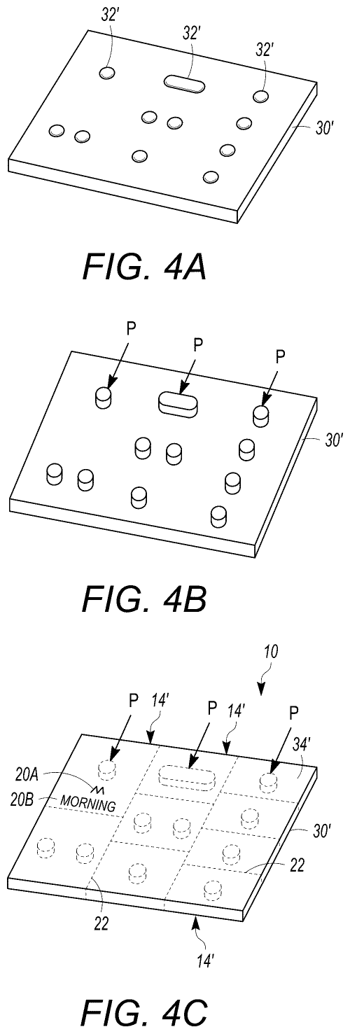

Referring to FIGS. 4A-C, another method of making the receptacle 10 of the present disclosure is generally illustrated. In this embodiment, the receptacle 10 includes the dosing sections 14′ of FIG. 2, with the embedded pharmaceuticals P. Again, the receptacle 10 is made using additive manufacturing, although other ways of making the receptacle are within the scope of the present disclosure. In the illustrated embodiment, at the start, the edible base 30′ (e.g., edible base layer) of the receptacle 10 is formed by the additive manufacturing process. The food grade 3D printer may form the edible base 30′ out of an edible material (e.g., a first edible material), such as one of the edible materials described herein. When forming the base 30′, one or more depressions or recesses 32′ may be formed for receiving the pharmaceuticals P. In this embodiment, each depression 32′ is sized and shape to correspond to the one pharmaceutical P the depression will receive. Because the receptacle 10 is being formed via additive manufacturing, each depression 32′can be individually tailored to the size and shape of the corresponding pharmaceutical P. The number and positioning of the depressions 32′ corresponds to the number and positioning of the dosing sections 14′.

After the base 30′ is formed, the pharmaceuticals P are placed on the base according to the treatment regimen, as illustrated in FIG. 4B. The pharmaceuticals P are arranged in the base 30′ according to the pharmaceutical doses required by the treatment regimen. Each pharmaceutical P is placed in one of the depressions 32′. In one embodiment, the depressions may be omitted the pharmaceuticals P may be placed directly on the surface of the base 30′. Placing of the pharmaceuticals P can occur manually or with one or more pharmaceutical placers/dispensers, as described above. After the pharmaceuticals P are placed, the rest of the receptacle 10 is formed on the base 30′. Generally speaking, an edible material (e.g., a second edible material) is added to the base to secure the pharmaceuticals P to the base 30′ (broadly, receptacle 10). This is done by adding the edible material to the base 30′ around the pharmaceuticals P, thereby embedding the pharmaceuticals in the edible material. Thus, broadly, the pharmaceuticals P are embedded in the edible material (e.g., first and second edible materials) of the receptacle 10. In one embodiment, the added edible material may contact the pharmaceuticals. In one embodiment, the added edible material is in the form of an edible covering layer 34′ (broadly, an edible cover) added to or formed directly on the base 30′ via the additive manufacturing process. Thus, the covering layer 34′ and the base 30′ may be considered a single, integrally formed component or body. The food grade 3D printer may form the covering layer 34′ out of an edible material (e.g., the second edible material), such as one of the edible materials described herein. The edible material of the covering layer 34′ may be the same or different than the edible material of the base 30′ (broadly, the first and second edible materials may be the same or different). In one embodiment, after the covering layer 34′ is applied, the lines of weakness 22 are formed. This can be done by scoring or kerfing the base 30′ and/or covering layer 34′ at the appropriate locations. In one embodiment, the covering layer 34′ and/or base 30′ are formed by the additive manufacturing process with the lines of weakness 22.

The card 12 is labeled with the dosing indicia 20. The dosing indicia 20 is applied to the card 12 to correspond to the dosing sections 14 (e.g., pharmaceutical doses). In one embodiment, the dosing indicia 20 is applied (e.g., printed by a food grade 3D printer) to the upper surface of the covering layer 34′. In one embodiment, the covering layer 34′ is formed with the dosing indicia 20 thereon (e.g., the dosing indicia is integral to the covering layer), as described above. In another embodiment, the dosing indicia 20 is applied by a grinder removing material from the covering layer 34′, as described above. Other ways of labeling the card with the dosing indicia are within the scope of the present disclosure. In the illustrated embodiment shown in FIG. 4C, only one set of dosing indicia 20 for one dosing section 14 is shown for clarity reasons, but it is understood each dosing section would include corresponding dosing indicia. After the receptacle 10 is completed (FIG. 4C), the receptacle can be packaged and transferred (e.g., shipped, transported, etc.) to the patient.

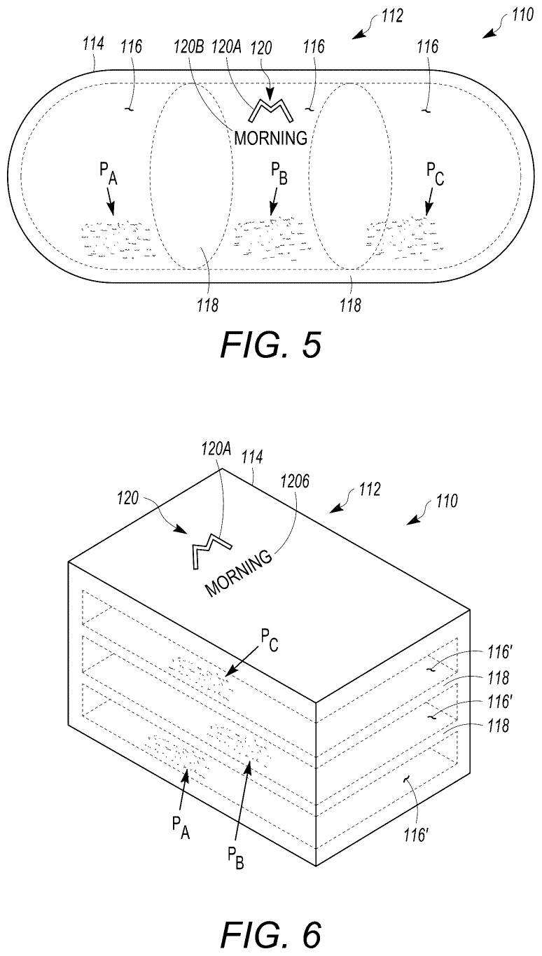

Referring to FIG. 5, another embodiment of an edible pharmaceutical receptacle of the present disclosure is generally indicated by reference numeral 110. The receptacle 110 is preferably organized by the treatment regimen (e.g., as prescribed by the medical professional, as customized for an individual patient, or otherwise). More particularly, the receptacle 110 organizes the plurality of pharmaceuticals P (e.g., a first pharmaceutical, a second pharmaceutical, a third pharmaceutical, etc.) for one pharmaceutical dose of the treatment regimen. In this embodiment, the receptacle 110 comprises an edible pharmaceutical carrier 112 having a bite-sized size and shape such that the carrier (broadly, the entire receptacle) can be eaten by a patient in one mouthful. In the illustrated embodiment, the carrier 112 has a size and shape similar to conventional pharmaceutical pills (e.g., a pill shape), although other configurations (e.g., sphere, pyramid, prism, hexahedron, etc.) are within the scope of the present disclosure. In this embodiment, the receptacle 110 corresponds to a dosing time (e.g., Monday morning) for the patient to take one more pharmaceuticals P of the treatment regimen. The receptacle 110 includes one pharmaceutical dose (e.g., the one or more pharmaceuticals P) of the treatment regimen for the specific dosing time. In one embodiment, the receptacle 110 is part of a group or collection of receptacles, with each receptacle containing one pharmaceutical dose of the treatment regimen. In other words, each receptacle corresponds to a different time (e.g., time of day and day of the week) the patient is to be administered pharmaceuticals P according to the treatment regimen. Accordingly, the group of receptacles 110 includes the plurality of pharmaceutical doses of the treatment regimen, with the number of receptacles corresponding to the number of pharmaceutical doses. The receptacles 110 in the group can be the same or different. For example, the pharmaceuticals P contained in one receptacle 110 may have a different amount relative to the another receptacle, and/or the pharmaceuticals contained in one receptacle may include one or more additional pharmaceuticals not included in another receptacle, and/or the pharmaceuticals contained in one receptacle may be the same as another receptacle, and/or one receptacle may have a different size and/or shape from another receptacle.

The edible pharmaceutical carrier 112 of the receptacle 110 includes an edible housing 114 comprising one or more walls. The housing 114 of the carrier 112 includes (e.g., defines) one or more compartments 116 (e.g., a first compartment, a second compartment, etc.). In the illustrated embodiment, the carrier 116 includes three compartments 116 (e.g., pharmaceutical compartments) although more or fewer compartments are within the scope of the present disclosure. Each compartment 116 is sized and shaped to contain the one or more pharmaceuticals P therein. Preferably, each compartment 116 holds only one pharmaceutical P. However, in other embodiments, one compartment 116 can hold multiple pharmaceuticals P if they are of a type that can be stored in contact with one another. The pharmaceuticals P are disposed in the compartments 116. In particular, the compartments 116 are enclosed and fully contain the pharmaceuticals P. This makes the pharmaceuticals P inaccessible to prevent the pharmaceutics from being removed from the carrier 112. For example, preferably the carrier 112 (e.g., housing 114) fully encases the pharmaceuticals P in the compartments 116 and the only way to access the pharmaceuticals is by breaking the carrier, such as what would occur when the carrier is eaten and chewed by the patient. In one embodiment, the size and shape of each compartment 116 can be conformed to the size and/or shape of the one or more pharmaceuticals P contained in the compartment. This way, the size of the compartment 116, and by extension the carrier 112, can be constructed to be just large enough to contain the pharmaceuticals P. This minimizes the amount of material needed to form the receptacle 110, keeping material costs down and minimizing manufacturing time. The compartments 116 can all be the same size and shape or different sizes and shapes. Where the housing 114 includes multiple compartments 116, the housing can include one or more partitions 118 in the interior of the housing for separating the compartments. Each compartment 116 may contain none, one, or more than one pharmaceutical P. In the illustrated embodiment, the carrier 112 contains three pharmaceuticals P_{A}, P_{B}, P_{C}, one in each compartment 116. The three pharmaceuticals are in the form of a powder. The powder form for the pharmaceuticals P may be preferred because it can be the purest form of the pharmaceutical and does not have any additional binder material and other fillers common in conventional pharmaceutical tablets and pills. In general, anywhere between 25%-75% of the material in conventional pharmaceutical tablets and pills can be binder and filler materials. Such binder and filler materials are not needed in the edible carrier 112 of the present disclosure. This allows the carrier 112 to be as small as possible by sizing the carrier to hold just the pharmaceutical P, not any binder and/or filler materials. It is understood the pharmaceuticals P may be in other forms (e.g., liquid, tablet, pill, etc.) as well. The pharmaceuticals P contained in the carrier 112 may be of the same form or different forms.

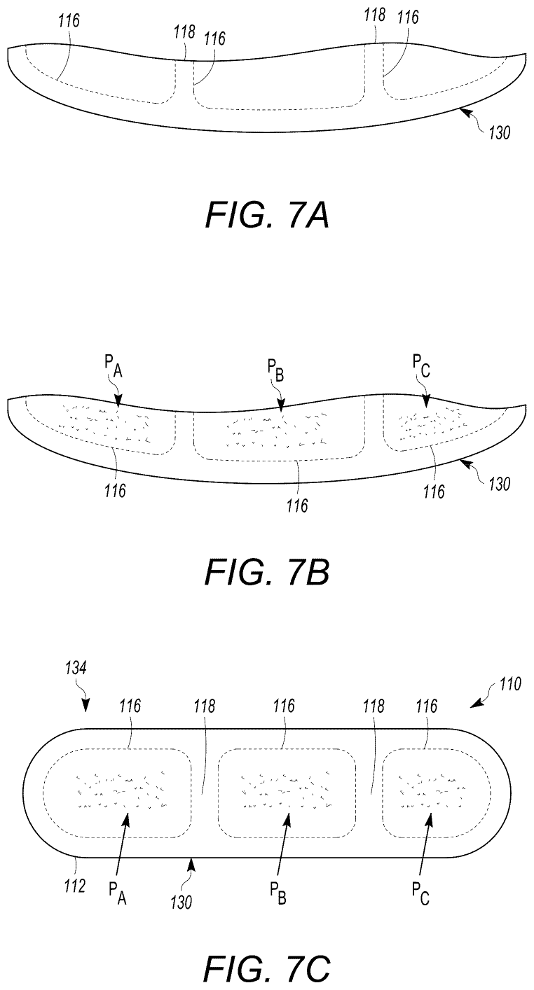

In the illustrated embodiment, the compartments 116 of the carrier 112 are arranged side-by-side. Other arrangements are within the scope of the present disclosure. For example, the compartments may be in a stacked arrangement, e.g., one on top of another. An example of such an embodiment is generally illustrated in FIG. 6. In this embodiment, the three compartments 116′ are arranged one on top of another such that the middle compartment overlies the bottom compartment and the top compartment overlies both the middle and bottom compartments. In one embodiment, the carrier can include a combination of side-by-side and stacked compartments. It is appreciated that the receptacle 110 of FIG. 6 is generally analogous to the receptacle 110 of FIG. 5, and thus the descriptions regarding the receptacle of FIG. 5 also apply to the receptacle of FIG. 6, except as clearly indicated or stated otherwise. For example, the carrier 112 of FIG. 6 has a different shape (e.g., a hexahedron shape) than the carrier 112 of FIG. 5.

In one embodiment, the pharmaceuticals P may be embedded into the carrier. Such an embodiment may be preferred if the pharmaceuticals are in a tablet or pill form. In this embodiment, the carrier may not include the compartments 116. Instead, the pharmaceuticals P are embedded into the body or edible material of the housing 14. Because the one or more pharmaceuticals P are embedded into the carrier the pharmaceuticals are held firmly in place and kept from contacting one another. The embedded pharmaceuticals P may have the same side-by-side or stacked arrangement as the compartments 116 described above. In one embodiment, the carrier includes a combination of compartments containing pharmaceuticals P and embedded pharmaceuticals.

Returning to FIG. 5, the receptacle 110 includes dosing indicia 120 associated therewith. The dosing indicia 120 can be part of the carrier 112 or otherwise associated with the receptacle 110. In the illustrated embodiment, the dosing indicia 120 is used to indicate a specific time to take (e.g., eat) the receptacle 110 and thereby take (e.g., ingest) the pharmaceuticals P contained in the carrier 112. The dosing indicia 120 includes a day indicia 120A and a day time indicia 120B (broadly, at least one of a day indicia or a day time indicia), as generally described above with respect to the day indicia 20A and the day time indicia 20B illustrated in FIG. 1. The dosing indicia 120 may be color to have a different appearance then the rest of the carrier 112. The dosing indicia 120 may be an integral part of the carrier 112 or be applied (e.g., printed) thereon. In other embodiments, the receptacle 110 is packaged in a container, such as a bag, pouch, blister pack, etc. which includes the dosing indicia. Other configurations of the dosing indicia are within the scope of the present disclosure and the dosing indicia may be used to convey other types of information, such as the pharmaceutical types, pharmaceutical size (e.g., 100 mg, 200 mg, etc.), etc. Preferably, the dosing indicia 120 is constructed of an edible material, such as the same edible material of the carrier 112. In some embodiments, the edible material of the dosing indicia is an edible ink.

The carrier 112 is constructed of one or more edible materials. In one embodiment, the edible materials are one or more of gelatin, hypromellose, makiid, and combinations thereof. Other suitable edible materials are within the scope of the present disclosure. As a result of making the carrier 112 out of edible materials, the entire carrier (broadly, receptacle 110) can be orally administered to or taken by the patient. This allows the patient to take the entire pharmaceutical dose of the treatment regimen in one bite by eating the entire receptacle 110 or to be swallowed in one gulp like a conventional pill. Keeping all the pharmaceuticals P together for the pharmaceutical dose ensures greater compliance to the treatment regimen by the patient. Moreover, it is recommended or required for certain pharmaceuticals P to be taken with food and eating the edible carrier 112 may satisfy this recommendation/requirement. The pharmaceuticals P are preferably in a form easy to ingest, such as a liquid, powder, or are chewable. However, the pharmaceuticals P may have other forms, such as swallowable tablets, as well. When it is time for a patient to take a pharmaceutical dose of the treatment regimen, the patient simply places the receptacle 110 in their mouth and eats the receptacle and the pharmaceuticals P contained therein. The patient takes all of the contents in the carrier 112. As mentioned above, the receptacle 110 (e.g., the carrier 112) has a bite-sized size and shape so that the entire receptacle can be eaten by the patient (e.g., interested into the patient's mouth) in one mouthful. If a patient were to eat the receptacle 110 in multiple bites, the breaking of the receptacle into smaller bites may inadvertently release the pharmaceuticals P from the carrier 112 before the carrier is completely eaten. In other words, the pharmaceuticals P may inadvertently fall out of the carrier 112. Keeping the receptacle 110 (e.g., carrier 112) small and bite-sized prevents such a situation from occurring.

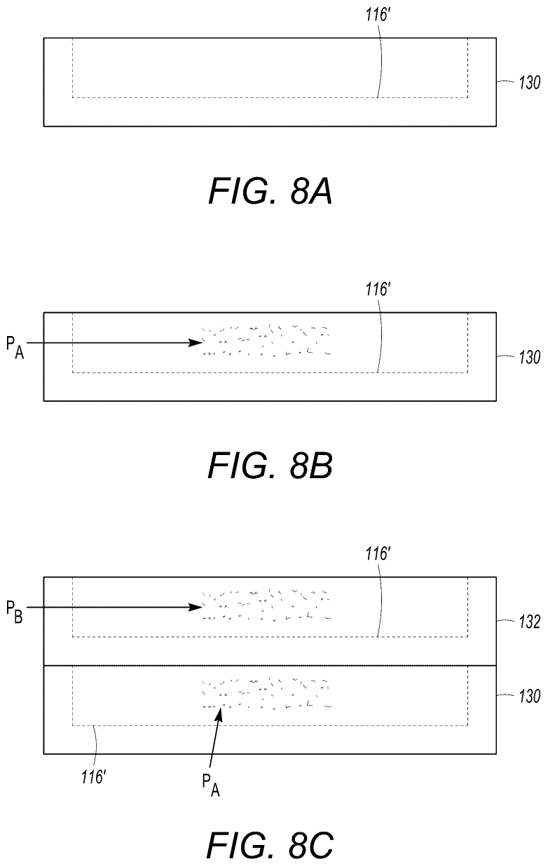

Referring to FIGS. 7A-C, one method of making the receptacle 110 of the present disclosure is generally illustrated. In this embodiment, the receptacle 110 is made using additive manufacturing (e.g., 3D printers), although other ways of making the receptacle are within the scope of the present disclosure. In one embodiment, the additive manufacturing process generally building the receptacle 110 from the bottom up. In the illustrated embodiment, at the start, a first or bottom portion 130 of the carrier 112 (broadly, receptacle 110) is formed by the additive manufacturing process. For example, a food grade or pharmaceutical grade 3D printer may form the bottom portion 130 out of an edible material (e.g., a first edible material), such as one of the edible materials described herein. When forming the bottom portion 130 of the carrier, the one or more compartments 116 of the carrier may also be formed (broadly, at least partially formed). In the illustrated embodiment, the compartments 116 are generally arranged side-by-side. As a result, the compartments 116 can be formed simultaneously with one another. The bottom portion 130 defines the one or more compartments 116 of the carrier 112 (broadly, defines at least a portion of the one or more compartments). In the illustrated embodiment, the bottom portion 130 defines a portion of each compartment 116 of the carrier 112. The compartments 116 (broadly, the carrier 112) may be sized and shaped to correspond to the size and shape needed to contain the specific quantity of pharmaceuticals P for the receptacle 110. In fact, because the receptacle 110 is being formed via additive manufacturing, the entire receptacle (including the size and shape of the compartments 116) can be customized to the treatment regimen of a particular patient. This minimizes the amount of material needed to form the receptacle, keeping material costs down and minimizing manufacturing time. In one example, the exterior shape of the receptacle 110 can be customized, such as to have a shape easy to ingest (e.g., oval, spherical, etc.). If needed, any partitions 118 can be added to the bottom portion 130 by the additive manufacturing process at this time.

After a sufficient portion of the compartments 116 are formed, the pharmaceuticals P are placed therein (e.g., on the bottom portion 130) according to the treatment regimen, as illustrated in FIG. 7B. The pharmaceuticals P are arranged in the base 30 according to the pharmaceutical dose of the treatment regimen the receptacle 110 will contain. Placing of the pharmaceuticals P can occur manually or with one or more pharmaceutical placers/dispensers, as described above. After the pharmaceuticals P are placed, the rest of the carrier 112 (broadly, receptacle 110) is formed on the bottom portion 130. Generally speaking, a second or upper portion 134 of the carrier 112 is added to the bottom portion 130. The additive manufacturing process adds edible material (e.g., a second edible material) to the bottom portion to finish building the carrier 112 and secure the pharmaceuticals P therein. The upper portion 134 closes the compartments 116. The upper portion 134 may also define a portion of each compartment 116 and may also include (e.g., define) a portion of any partitions 118. In one embodiment, the upper portion 134 is formed separately of the bottom portion 130 and then attached to the bottom portion, such as by ultrasonic welding. In this embodiment, the respective upper and bottom portions 134, 130 may have corresponding projections and recesses sized and shaped to mate with one another to assist in the alignment of the upper portion on the bottom portion. In another embodiment, the upper portion 134 is formed directly on the bottom portion 130 using the additive manufacturing process. In either case, a food grade 3D printer may form the upper portion 134 out of an edible material (e.g., the second edible material), such as one of the edible materials described herein. The edible material of the upper portion 134 may be the same or different than the edible material of the bottom portion 130 (broadly, the first and second edible materials may be the same or different).

The carrier 112 is labeled with the dosing indicia 120. In one embodiment, the dosing indicia 120 can be applied (e.g., printed by a food grade 3D printer) to generally any portion of the carrier 112 (e.g., the bottom portion 130, the upper portion 134). In one embodiment, the carrier 112 (e.g., the bottom portion 130, the upper portion 134) is formed with the dosing indicia 120 thereon (e.g., the dosing indicia is integral to the housing 114 of the carrier). For example, the food grade 3D printer may form the dosing indicia 120 as the printer is forming the carrier, such as by printing additional edible material into the housing 114. In another embodiment, the dosing indicia 120 is applied by a grinder (controlled by a CNC machine) which removes material from the housing 114 of the carrier 112 to form the dosing indicia. Other ways of labeling the carrier with the dosing indicia are within the scope of the present disclosure. After the receptacle 110 is completed (FIG. 7C), the receptacle can be packaged and transferred (e.g., shipped, transported, etc.) to the patient. This may include packaging the receptacle 110 with other receptacles of the pharmaceutical order and shipping all the receptacles of the pharmaceutical order together to the patient.

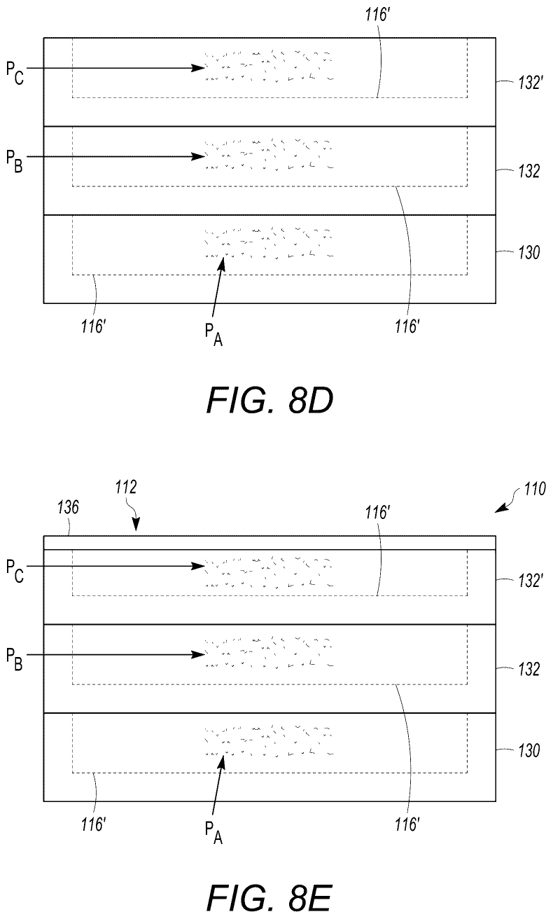

Referring to FIGS. 8A-E, another method of making the receptacle 110 of the present disclosure is generally illustrated. In this embodiment, the receptacle 110 includes the compartments 116′ of FIG. 6 that are in a stacked arrangement. Again, the receptacle 110 is made using additive manufacturing, although other ways of making the receptacle are within the scope of the present disclosure. In the illustrated embodiment, at the start (FIG. 8A), a first or bottom potion 130 of the carrier 112 (broadly, receptacle 110) is formed by the additive manufacturing process. The food grade 3D printer may form bottom portion 130 out of an edible material (e.g., a first edible material), such as one of the edible materials described herein. The bottom potion 130 is formed with one of the compartments 116′ therein (broadly, at least a portion of the one compartment). As explained above, the compartment 116′ can be sized and shaped to the size and shape needed to contain the specific quantity of pharmaceuticals P for the receptacle 110. In fact, because the receptacle 110 is being formed via additive manufacturing, the entire receptacle (including the size and shape of the compartments 116′) can be customized to the treatment regimen of a particular patient. This minimizes the amount of material needed to form the receptacle, keeping material costs down and minimizing manufacturing time. If needed, any vertical partitions can be added to the bottom portion 130 by the additive manufacturing process at this time. After the bottom portion 130 is formed, one of the pharmaceuticals P is placed therein, as illustrated in FIG. 8B, according to the treatment regimen. It is understood, more than one pharmaceutical P can be placed in the compartment 116′. Placing of the pharmaceuticals P can occur manually or with one or more pharmaceutical placers/dispensers, as described above.

Referring to FIG. 8C, after the pharmaceutical P is placed, the next portion of the carrier 112 is formed on the bottom portion 130. In the illustrated embodiment, a second or intermediate portion 132 of the carrier 112 is formed by the additive manufacturing process. The intermediate portion 132 is formed on (e.g., arranged on top of) the bottom portion 130. Generally speaking, the intermediate portion 132 of the carrier 112 is added to the bottom portion 130. The additive manufacturing process adds edible material (e.g., a second edible material) to the bottom portion to finish building the carrier 112 and secure the pharmaceutical P therein. The intermediate portion 132 closes the compartment 116′ of the bottom portion 130. The intermediate portion 132 may also define a portion of the compartment 116′ of the bottom portion 130 and may also include (e.g., define) a portion of any partitions 118. The intermediate portion 132 also includes (e.g., is formed with) one of the compartments 116′ (broadly, at least a portion of the one compartment), like the bottom portion 130 as described above. In one embodiment, the intermediate portion 132 is formed separately of the bottom portion 130 and then attached to the bottom portion, such as by ultrasonic welding. In this embodiment, the respective intermediate and bottom portions 132, 130 may have corresponding projections and recesses sized and shaped to mate with one another to assist in the alignment of the upper portion on the bottom portion. In another embodiment, the intermediate portion 132 is formed directly on the bottom portion 130 using the additive manufacturing process. In either case, a food grade 3D printer may form the intermediate portion 132 out of an edible material (e.g., the second edible material), such as one of the edible materials described herein. The edible material of the intermediate portion 132 may be the same or different than the edible material of the bottom portion 130 (broadly, the first and second edible materials may be the same or different). After the intermediate portion 132 is formed, one of the pharmaceuticals P is placed therein, as illustrated in FIG. 8C, according to the treatment regimen. It is understood, more than one pharmaceutical P can be placed in the compartment 116′. Placing of the pharmaceuticals P can occur manually or with one or more pharmaceutical placers/dispensers, as described above.

Referring to FIG. 8D, in the illustrated embodiment, the carrier 112 includes a third portion or another intermediate portion 132′, which is generally the same as the intermediate portion 132 described above except that the additional intermediate portion 132′ is arranged on top of the previous intermediate portion 132. Thus, the descriptions regarding the previous intermediate portion 132 also apply to the additional intermediate portion 132′. For example, the additional intermediate portion can be made out of an edible material (e.g., a third edible material), such as one of the edible materials described herein, and the edible material can be the same or different as the edible materials of the intermediate portion 132 and/or bottom portion 130. After the additional intermediate portion 132 is formed, one or more of the pharmaceuticals P is placed therein, as illustrated in FIG. 8D, according to the treatment regimen as described above. More additional intermediate sections 132′ can be added as needed in order to contain all the pharmaceuticals P of the pharmaceutical dose.

Referring to FIG. 8E, after all the intermediate sections 132, 132′ are formed, an edible cover or lid 136 is added to the top. In one embodiment, the edible cover 136 is formed separately and then attached to the rest of the carrier 112 (e.g., the top most intermediate section 132, 132′), such as by ultrasonic welding. In this embodiment, the cover 136 and the top most intermediate section 132, 132′ may have corresponding projections and recesses sized and shaped to mate with one another to assist in the alignment of the cover on the rest of the carrier 112. In another embodiment, the edible cover 136 is formed directly on the rest of the carrier 112 using the additive manufacturing process. In either case, a food grade three dimensional (“3D”) printer may form the edible cover 136 out of an edible material (e.g., a third edible material), such as one of the edible materials described herein, and the edible material can be the same or different as the edible materials of the rest of the carrier 112. The edible cover 136 closes the compartment 116′ of the top most intermediate section 132, 132′. After, the carrier 112 is labeled with the dosing indicia 120, as described above. After the receptacle 110 is completed (FIG. 8E), the receptacle can be packaged and transferred (e.g., shipped, transported, etc.) to the patient. This may include packaging the receptacle 110 with other receptacles of the pharmaceutical order and shipping all the receptacles of the pharmaceutical order together to the patient.

The pharmaceuticals P embedded in the sections 14 of the pharmaceutical card 12 can be in the solid form from the drug manufacturer. The pharmaceuticals P are organized per dosing time and set in the appropriate section of the pharmaceutical card 12. In another example embodiment, the drugs are in a powered form or small form that is measured and placed in a section of the pharmaceutical card 12.

In an example embodiment, a pharmaceutical additive printer and/or a pharmaceutical dispenser is adjacent the food grade 3D printer and can alternative operate with the food grade 3D printer to input the pharmaceutical into the food grade matrix. Alternative the food grade 3D printer can create a well for the pharmaceutical and stop. Thereafter, the pharmaceutical additive printer and/or pharmaceutical dispenser can deposit the pharmaceutical into the well. After the pharmaceutical is deposited in the well (and after confirmed by a light detector or scale on which the partial carrier 12 is being built by the printer) by the pharmaceutical additive printer and/or pharmaceutical dispenser, the food grade 3D printer will encase the pharmaceutical to complete the carrier 12.

While various embodiments described herein describe building a carrier with a pharmaceutical, other ingestibles can be used the additive building of a carrier. Other example ingestibles can include vitamins, supplements, or other medically related substances.

It is apparent that the elements, features, and/or teachings set forth in each embodiment disclosed herein are not limited to the specific embodiment(s) in which the elements, features and/or teachings are explicitly described. Accordingly, it is understood that the elements, features and/or teachings described in one embodiment may be applied to one or more of the other embodiments disclosed herein, even if said elements, features and/or teachings where not described herein as being a part of said one or more of the other embodiments.

The Title, Field, and Background are provided to help the reader quickly ascertain the nature of the technical disclosure. They are submitted with the understanding that they will not be used to interpret or limit the scope or meaning of the claims. They are provided to introduce a selection of concepts in simplified form that are further described in the Detailed Description. The Title, Field, and Background are not intended to identify key features or essential features of the claimed subject matter, nor is it intended to be used as an aid in determining the claimed subject matter.

When introducing elements of aspects of the disclosure or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that several advantages of the aspects of the disclosure are achieved and other advantageous results attained.

Not all of the depicted components illustrated or described may be required. In addition, some implementations and embodiments may include additional components. Variations in the arrangement and type of the components may be made without departing from the spirit or scope of the claims as set forth herein. Additional, different or fewer components may be provided and components may be combined. Alternatively or in addition, a component may be implemented by several components.

The above description illustrates the aspects of the disclosure by way of example and not by way of limitation. This description enables one skilled in the art to make and use the aspects of the disclosure, and describes several embodiments, adaptations, variations, alternatives and uses of the aspects of the disclosure, including what is presently believed to be the best mode of carrying out the aspects of the disclosure. Additionally, it is to be understood that the aspects of the disclosure is not limited in its application to the details of construction and the arrangement of components set forth in the description or illustrated in the drawings. The aspects of the disclosure are capable of other embodiments and of being practiced or carried out in various ways. Also, it will be understood that the phraseology and terminology used herein is for the purpose of description and should not be regarded as limiting.

Having described aspects of the disclosure in detail, it will be apparent that modifications and variations are possible without departing from the scope of aspects of the disclosure as defined in the appended claims. It is contemplated that various changes could be made in the above constructions, products, and methods without departing from the scope of aspects of the disclosure. In the preceding specification, various embodiments have been described with reference to the accompanying drawings. It will, however, be evident that various modifications and changes may be made thereto, and additional embodiments may be implemented, without departing from the broader scope of the aspects of the disclosure as set forth in the claims that follow. The specification and drawings are accordingly to be regarded in an illustrative rather than restrictive sense.

What is claimed is:

1. An edible pharmaceutical receptacle comprising:
a plurality of pharmaceuticals including a first pharmaceutical and second pharmaceutical, the second pharmaceutical being different than the first pharmaceutical;
an edible pharmaceutical card including a plurality of edible dosing sections joined together, the plurality of edible dosing sections including a first edible dosing section directly joined to a second edible dosing section, the first and second edible dosing sections arranged to be manually broken apart to separate the first and second edible dosing sections from one another, the first edible dosing section containing the first pharmaceutical of the plurality of pharmaceuticals and the second edible dosing section containing the second pharmaceutical of the plurality of pharmaceuticals.

2. The edible pharmaceutical receptacle of claim 1, wherein the first edible dosing section defines a first dosing compartment, the first pharmaceutical disposed in the first dosing compartment, and wherein the second edible dosing section defines a second dosing compartment, the second pharmaceutical disposed in the second dosing compartment.

3. The edible pharmaceutical receptacle of claim 1, wherein the first pharmaceutical is embedded in the first edible dosing section, and wherein the second pharmaceutical is embedded in the section edible dosing section.

4. The edible pharmaceutical receptacle of claim 1, wherein the edible pharmaceutical card is constructed of one or more edible materials.

5. The edible pharmaceutical receptacle of claim 1, wherein the edible pharmaceutical card comprises an edible material selected from the group consisting of gelatin, hypromellose, makiid, sugar, edible paper, beeswax, MonoSol's film, and combinations thereof.

6. A method of making an edible pharmaceutical receptacle comprising:
forming, via additive manufacturing, an edible base of an edible pharmaceutical receptacle using a first edible material;
placing a plurality of pharmaceuticals on the edible base in a plurality of pharmaceutical doses, the plurality of pharmaceutical doses includes a first pharmaceutical dose and a second pharmaceutical dose, the first pharmaceutical dose containing one or more pharmaceuticals of the plurality of pharmaceuticals, the second pharmaceutical dose containing one or more pharmaceuticals of the plurality of pharmaceuticals; and
adding a second edible material, via additive manufacturing, to the edible base to secure the plurality of pharmaceuticals to the edible pharmaceutical receptacle in the plurality of pharmaceutical doses.

7. The method of claim 6, wherein said adding the second edible material includes embedding the plurality of pharmaceuticals in the second edible material.

8. The method of claim 6, wherein said forming the edible base includes forming a plurality of dosing compartments in the edible base, and wherein said placing the plurality of pharmaceuticals includes placing each pharmaceutical dose in one of the plurality of dosing compartments, and wherein said adding the second edible material includes closing the plurality of dosing compartments.

9. The method of claim 6, wherein the first and second edible materials are the same or different.

10. The method of claim 6, further comprising labeling the edible pharmaceutical receptacle with dosing indicia associated with each pharmaceutical dose of the plurality of pharmaceutical doses, each dosing indicia including at least one of a day indicia indicating a day the corresponding pharmaceutical dose is to be taken or a day time indicia indicating a time of day the corresponding pharmaceutical dose is to be taken.

11. An edible pharmaceutical receptacle comprising:
a plurality of pharmaceuticals including a first pharmaceutical and second pharmaceutical;
an edible pharmaceutical carrier having a bite-sized size and shape such that the edible pharmaceutical carrier can be eaten by a patient in one mouthful, the edible pharmaceutical carrier including an enclosed first compartment, an enclosed second compartment, and a partition separating the first and second compartments, the first pharmaceutical disposed in the first compartment and the second pharmaceutical disposed in the second compartment.

12. The edible pharmaceutical receptacle of claim 11, wherein the first and second compartments are arranged side-by-side.

13. The edible pharmaceutical receptacle of claim 11, wherein the first and second compartment are arranged on top of one another.

14. The edible pharmaceutical receptacle of claim 11, wherein the first pharmaceutical and the second pharmaceutical are each in a powder form.

15. The edible pharmaceutical receptacle of claim 11, wherein the edible pharmaceutical carrier is constructed of one or more edible materials.

16. The edible pharmaceutical receptacle of claim 11, wherein the edible pharmaceutical card comprises an edible material selected from the group consisting of gelatin, hypromellose, makiid, sugar, edible paper, beeswax, MonoSol's film, and combinations thereof.

17. A method of making an edible pharmaceutical receptacle comprising:
forming, via additive manufacturing, a first portion of the edible pharmaceutical receptacle using a first edible material, said first portion defining a first compartment;
placing a first pharmaceutical in the first compartment; and
forming, via additive manufacturing, a second portion of the edible pharmaceutical receptacle using a second edible material, said second portion closing the first compartment.

18. The method of claim 17, wherein the second portion defines a second compartment, the method further comprising placing a second pharmaceutical in the second compartment.

19. The method of claim 18, wherein said forming the second portion includes arranging the second portion on top of the first portion.

20. The method of claim 17, further comprising labeling the edible pharmaceutical receptacle with dosing indicia, the dosing indicia including at least one of a day indicia indicating a day the corresponding pharmaceutical dose is to be taken or a day time indicia indicating a time of day the corresponding pharmaceutical dose is to be taken.

21. The method of claim 17, wherein the first and second edible materials are the same or different.
